(19) United States
(12) Plant Patent Application Publication
(10) Pub. No.: US 2009/0100556 P1
(43) Pub. Date: Apr. 16, 2009

(54) Syzygium smithii plant named 'DOW30'

(76) Inventor: Warren Downes, Theresa Park (AU)

Correspondence Address: Sam R. McCoy Jr., P.O. Box 2108, Mount Pleasant, SC, 29465, US

(21) Appl. No.: 11/903,037

(22) Filed: Sep. 20, 2007

(51) Int. Cl. A01H 5/00

(52) U.S. Cl. PLT/216

(57) ABSTRACT

‘DOW30’ is a distinctive variety of Syzygium smithii, which is characterized by its light green immature leaf and stem colors during a new flush of shoot growth.

### LATIN NAME OF THE GENUS AND SPECIES

The Latin name of the novel variety disclosed herein is Syzygium smithii ‘DOW30’.

Prior to a taxonomic review in 2006 the Latin name was Acmena smithii (Reference: Craven, L. A., Biffin, E., Ashton, P. S. (2006). Acmena, Acmenospora, Cleistocalyx, Piliocalyx and Waterhousea formally transferred to Syzygium (Myrtaceae). Blumea 51: 131-142.

### VARIETY DENOMINATION

The inventive variety of Syzygium smithii disclosed herein has been given the variety denomination ‘DOW30’.

### BACKGROUND OF THE INVENTION

The present invention relates to a new and distinct variety of evergreen perennial Syzygium smithii, which has been named ‘DOW30’. Syzygium smithii is a small to large Australian native tree that is used as an ornamental tree in landscape and amenity horticulture. Syzygium smithii has a broad, bushy plant growth habit with a squat appearance. Glossy green leaves are arranged opposite and the new flush of growth is green colored instead of the usual reddish coloration.

An application for Plant Breeders Rights for ‘DOW30’ has been lodged with the Australian Plant Breeders Rights Office, and was accepted on 29 Apr. 2006 (under Application No. 2005/317).

Parentage: The cultivar ‘DOW30’ was discovered in 2003 in Tuckombil, New South Wales, Australia, during a seedling selection of cultivated open pollinated common un-named species form of Syzygium smithii (unpatented). The parent form is characterized by a medium bushy plant growth habit combined with a reddish immature leaf and stem colors during a new flush of shoot growth. Selection criteria for ‘DOW30’ was a light green immature leaf color during a new flush of shoot growth.

Asexual reproduction. The new variety ‘DOW30’ was first asexually propagated by vegetative cuttings in the state of New South Wales, Australia in 2003. The distinctive characteristics of cultivar ‘DOW30’ have remained stable and true to type through successive cycles of asexual propagation.

### SUMMARY OF THE INVENTION

‘DOW30’ is a distinctive variety of Syzygium smithii, which is characterized by its light green immature leaf and stem colors during a new flush of shoot growth.

### BRIEF DESCRIPTION OF THE DRAWINGS

FIG. 1 shows a Syzygium smithii ‘DOW30’ plant at approximately 24-months of age. The growth habit is broad and squat with dense foliage.

FIG. 2 shows the green color of young leaves and stem in a flush of new growth of ‘DOW30’.

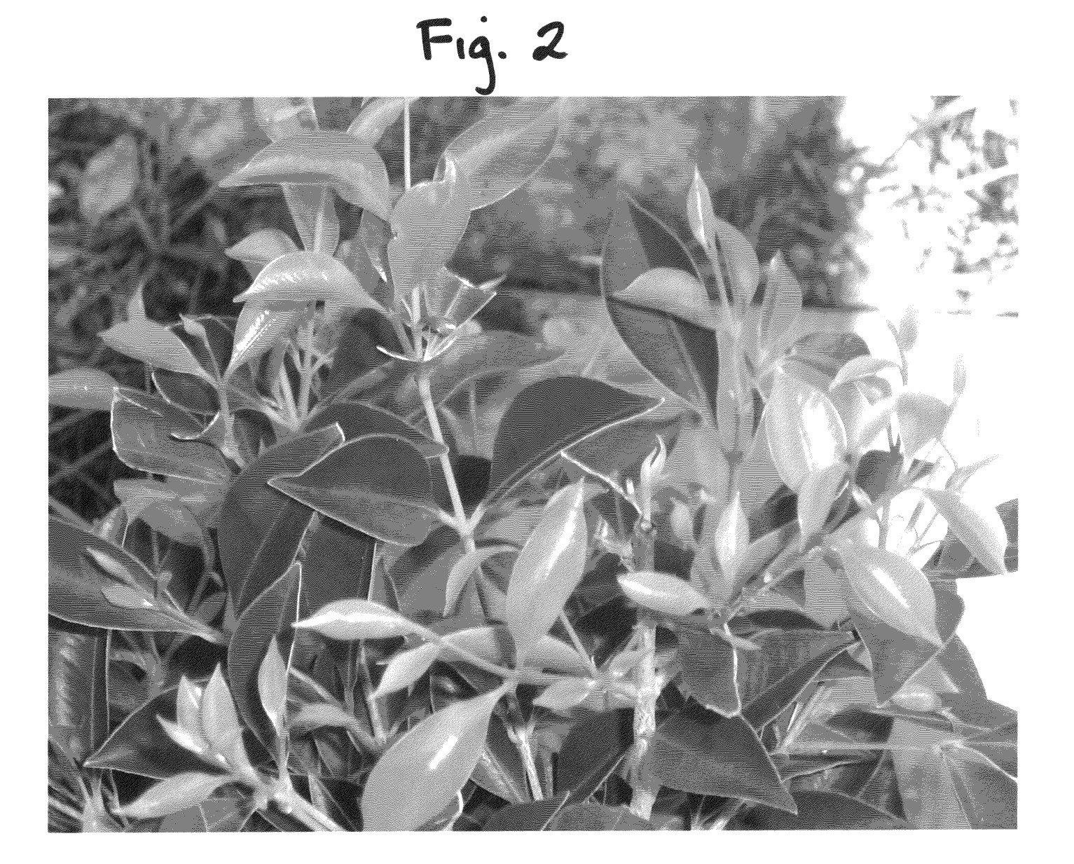

FIG. 3 shows the new foliage and bushy habit of ‘DOW30’.

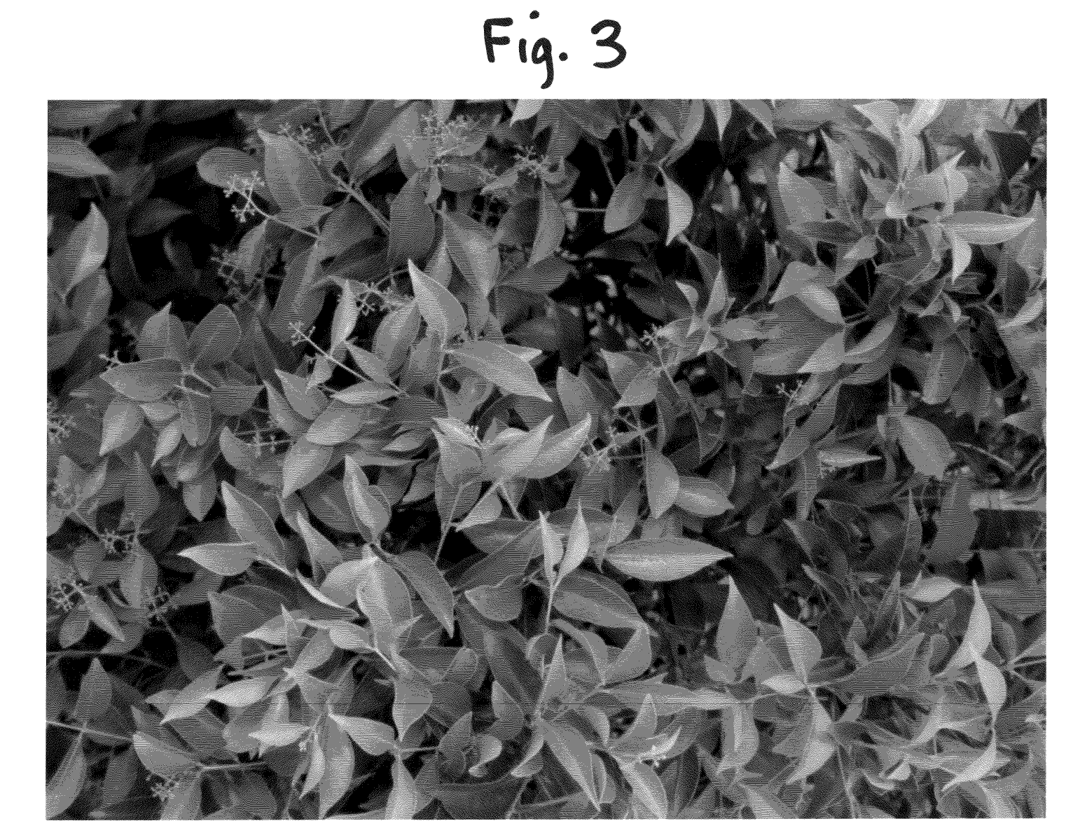

FIG. 4 shows the new foliage and the inflorescence structure (panicle) of ‘DOW30’

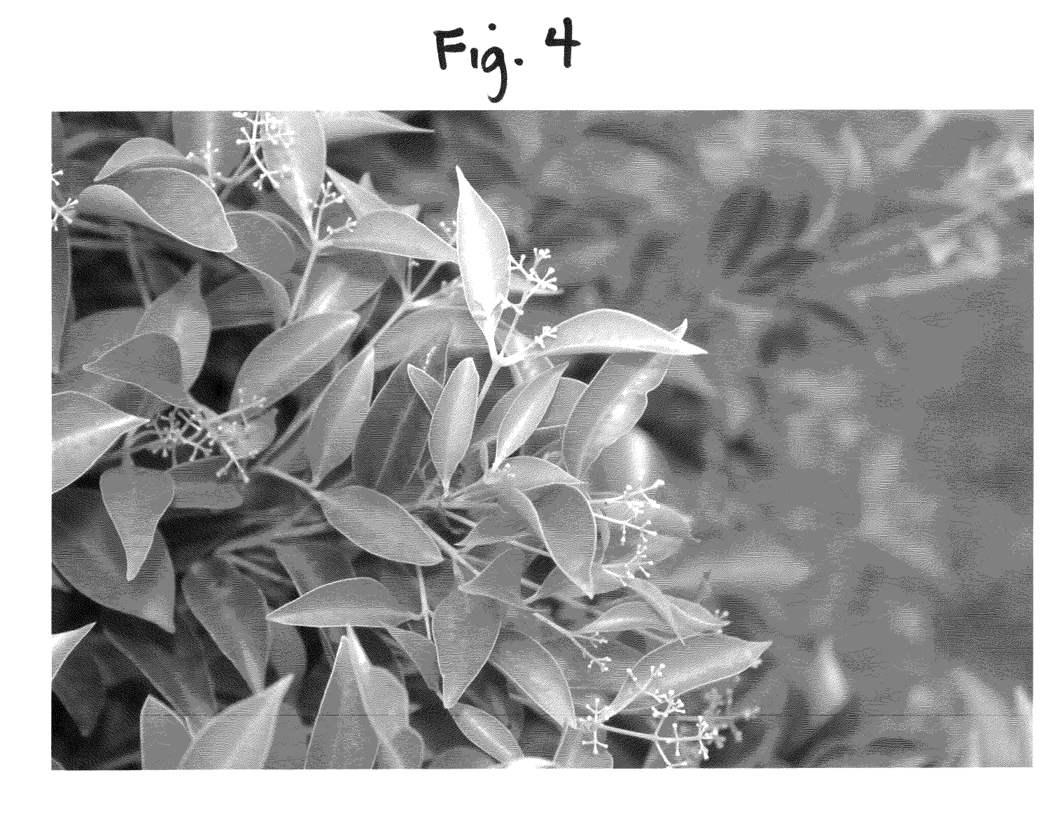

### DETAILED BOTANICAL DESCRIPTION OF THE VARIETY

The following is a detailed botanical description of a new and distinct variety of Syzygium smithii known as ‘DOW30’ based upon observations of 24-month old plants grown in nursery pots in full sun in open beds in Theresa Park, New South Wales, Australia during 2005- 2006 (as described below in connection with Table 1). Plant observations and descriptions were taken in winter-spring 2006.

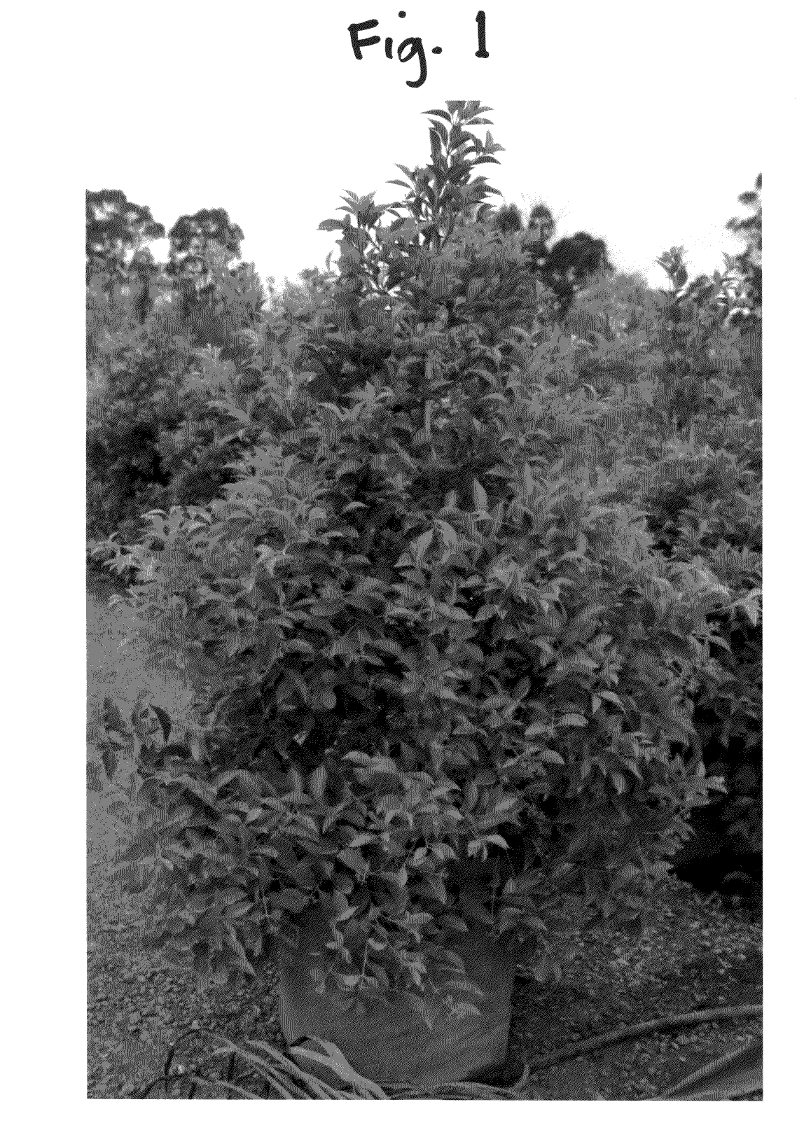

Those skilled in the art will appreciate that certain characteristics will vary with older or, conversely, with younger plants. ‘DOW30’ has not been observed under all possible environmental conditions. Where dimensions, sizes, colors and other characteristics are given, it is to be understood that such characteristics are approximations or averages set forth as accurately as practicable. The phenotype of the variety may differ from the descriptions set forth herein with variations in environmental, climactic and cultural conditions. Color notations are based on The Royal Horticultural Society Colour Chart, The Royal Horticultural Society, London, 1995 edition. ‘DOW30’ is an evergreen perennial Syzygium smithii. ‘DOW30’ is a bushy tree with a squat growth habit that is broad for its height and a vigorous growth rate. ‘DOW30’ has an elliptic leaf shape with an acuminate leaf apex shape, an attenuate leaf base, an entire leaf margin. The leaf is slightly concave in cross-section and slightly recurved along the longitudinal axis (mid rib). The surface of the leaf upper side of ‘DOW30’ has a glossy, dark green appearance and an absence of pubescence. The surface of the leaf lower side of ‘DOW30’ has a dull, lighter green appearance and an absence of pubescence. ‘DOW30’ has a light green immature leaf color light (expanding new growth). ‘DOW30’ has a green new stem color. These characteristics are unusual for Syzygium smithii, as these plants usually have a reddish immature leaf and stem colors (new growth flush). A botanical description of ‘DOW30’ and comparisons with the parent form of Syzygium smithii are provided below.
Technical description of the variety:
Plant characteristics: Growth habit upright bushy and squat (broad for its height), height medium (average 2 metres) and spread broad (average 1.2 m) for a 3 year old plant grown in a 45 L container (not yet recorded for a fully mature tree).
Stems: new stem on soft shoot tips has a smooth, glossy surface texture, color of young stem yellow green (RHS 144B), gradually changing with age to yellow green (RHS 144A). The stem color of semi-hardened growth is grayed orange (between RHS 164A and RHS 164C and between RHS 165A and 165C) with hints of gray (RHS 201D). The stem color of hardened growth is gray brown circa RHS 199D to gray RHS 201D. The trunk bark's surface has a finely rough texture which may crack with age and is colored gray brown ca RHS 199D.
Leaves: Attitude horizontal to erect, mature length medium (range 5.5 to 6.5 cm), mature width medium (range 20 to 25 mm), emerging leaf color yellow green (RHS 144A), fading towards margin and becoming slightly translucent, immature leaf upper side color yellow green (RHS 144A) with strong surface glossiness, mature leaf upper side color yellow green (ca RHS 147A) mature leaf lower side color yellow green (RHS 146B), variegation absent, shape elliptic, apex acuminate, leaf base attenuate, cross section slightly concave and slightly recurved along the longitudinal axis (mid rib), margin entire, upper side midrib color yellow green (RHS 144A-B), lower side midrib color yellow green (RHS 146B) blends in with leaf blade, venation pinnate and corresponding to leaf color, petiole length short-medium (range 5-7 mm), petiole color yellow green (RHS 144B), petiole texture finely wrinkled and weakly glossy.
Flowers and fruiting:
Inflorescence: ‘DOW30’ has a medium inflorescence size of length range 30 to 50 mm and width range 25-40 mm. The inflorescence is a panicle and is displayed terminally from just within to just beyond the foliage. Flowers last approximately 2 to 3 weeks in Sydney, New South Wales, Australia and flowering season is late spring to early summer. Fragrance is present and weak to medium as typical of the species. The peduncle is of a medium length (range 55-65 mm) and peduncle color is approximately yellow-green RHS 144A-B. FIG. 1 shows a ‘DOW30’ plant demonstrating the prominence of the inflorescences.
Flowers: Flowers are single and numerous ranging from 20-24 per inflorescence. Petals are insignificant, typical of the species. Stamens are numerous (more than 15) and prominent, length 5-8 mm, colored white corresponding approximately to RHS 155D. Pistil is single and positioned within the stamens. Pedicel is short with length 2-3 mm and color approximately yellow-green RHS 144A-B.
Fruit: Fruit is a berry, diameter range 10-15 mm, shape is globose, color is red purple approximately RHS 59B-C.
Cold and heat tolerance: ‘DOW30’ has not yet been observed under all conditions. ‘DOW30’ is heat tolerant. It adapted well to the high heat conditions greater than 40 degrees Celsius during summer in Theresa Park, New South Wales, Australia without any noticeable damage. It has survived frosts to minus 4 degrees Celcius. Testing for environmental tolerances is ongoing.
Drought tolerance: Mild drought tolerance typical of the species once established. Prefers moist condition, can survive wet conditions with adequate drainage, but not boggy conditions. ‘DOW30’ has not yet been observed under all conditions. Testing for environmental tolerances is ongoing.
Pest and disease resistance: No pests or diseases observed to date on unstressed plants.
Cultural conditions: ‘DOW30’ can tolerate low nutrient conditions; it does not like continually wet soil conditions, but can tolerate well-draining sandy soils to very heavy clay soils.

### Comparisons with other Syzygium smithii.

‘DOW30’ is a more attractive ornamental tree as compared with the parent Syzygium smithii (unpatented, wild type). ‘DOW30’ is characterized by its light green immature leaf and stem colors during a new flush of shoot growth as compared with Syzygium smithii. The appearance of ‘DOW30’ is green during a flush of new growth, whereas Syzygium smithii has reddish immature leaf and stem colors in the new growth flush (see Table 1).

The Syzygium smithii (unpatented, wild type) is the most similar comparator variety to ‘DOW30’. All known cultivars have reddish colored new growth flush.

A comparative trial of Syzygium cultivar ‘DOW30’ with Syzygium smithii (unpatented, wild type) is currently being carried out in Theresa Park, New South Wales, Australia. The initial data are presented in Table 1 below.

TABLE 1

| | Syzygium Variety | |
|---|---|---|
| Characteristic | ‘DOW30’ | Syzygium smithii |
| Plant: growth habit | Squat: short height | medium height |
| | to broad width | and width |
| Stem: new color | green | reddish |
| Leaf: immature color | green | reddish |

That which is claimed is:

1. A new and distinct variety of Syzygium smithii plant named ‘DOW30’, substantially as described and illustrated herein.
